(19) United States
(12) Patent Application Publication
(10) Pub. No.: US 2014/0080889 A1
(43) Pub. Date: Mar. 20, 2014

(54) Statin and Omega-3 Fatty Acids for Lipid Therapy

(71) Applicant: GlaxoSmithKline, LLC, Philadelphia, PA (US)

(72) Inventors: George BOBOTAS, Tarpon Springs, FL (US); Roelof M. Rongen, Caliton, NJ (US); Egil Bodd, Oslo (NO); Hogne Vik, Eiksmarka (NO)

(73) Assignee: GlaxoSmithKline, LLC, Philadelphia, PA (US)

(21) Appl. No.: 14/088,621

(22) Filed: Nov. 25, 2013

Related U.S. Application Data
(63) Continuation of application No. 12/977,109, filed on Dec. 23, 2010, now abandoned.
Application No. 12/977,109 is a continuation of application No. 12/256,806, filed on Oct. 23, 2008, now abandoned.
Application No. 12/256,806 is a continuation-in-part of application No. 11/742,292, filed on Apr. 30, 2007, now abandoned.
Application No. 11/742,292 is a continuation-in-part of application No. 11/284,095, filed on Nov. 22, 2005, now abandoned.

(51) Int. Cl. A61K 31/40; A61K 31/202; A61K 31/351

(52) U.S. Cl. CPC A61K 31/40; A61K 31/351; A61K 31/202
USPC 514/423

(57) ABSTRACT

A method and composition for lipid therapy. In some embodiments, the method comprises providing a subject group having a baseline triglyceride level of 200 to 499 mg/dl and being at or near its low-density lipoprotein cholesterol (LDL-C) level goal, and reducing the triglyceride level and the non-high-density lipoprotein cholesterol (non-HDL-C) level of the subject group as compared to treatment with a 3-hydroxy-3-methyl glutaryl coenzyme A (HMG CoA) inhibitor alone, by administering to the subject group an effective amount of an HMG CoA inhibitor and a composition comprising omega-3 fatty acids.

The present application is a divisional of Ser. No. 12/977,109, filed Dec. 23, 2010 (pending), which is a continuation of application Ser. No. 12/256,806, filed Oct. 23, 2008 (now abandoned), which is a continuation-in-part of application Ser. No. 11/742,292, filed Apr. 30, 2007 (now abandoned), which is a continuation-in-part of application Ser. No. 11/284,095 (now abandoned), filed Nov. 22, 2005, which claims priority from provisional patent application Ser. No. 60/633,125, filed Dec. 6, 2004, Ser. No. 60/659,099, filed Mar. 8, 2005, and Ser. No. 60/699,866, filed Jul. 18, 2005. The present application also claims priority from provisional patent application Ser. No. 60/840,012, filed Aug. 25, 2006, Ser. No. 60/850,280, filed Oct. 10, 2006, and Ser. No. 60/852,398, filed Oct. 18, 2006. The disclosure of the parent and priority applications is hereby incorporated by reference.

### FIELD OF THE INVENTION

The present invention relates to a method utilizing simultaneous or concomitant administration, e.g. a single or separate administration or a unit dosage, of a combination of an HMG-CoA inhibitor and omega-3 fatty acids for the treatment of patients with hypertriglyceridemia or hypercholesterolemia or mixed dyslipidemia, coronary heart disease (CHD), cardiovascular disease, vascular disease, atherosclerotic disease and related conditions, and for the prevention or reduction of cardiovascular, cardiac, and vascular events.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (TC), LDL-C, and apolipoprotein B (Apo-B, a membrane complex for LDL-C and VLDL-C) promote human atherosclerosis and decreased levels of HDL-C and its transport complex, apolipoprotein A (Apo-A), which are associated with the development of atherosclerosis. Further, cardiovascular morbidity and mortality in humans can vary directly with the level of TC and LDL-C and inversely with the level of HDL-C. In addition, researchers have found that non-HDL cholesterol (non-HDL-C) is an important indicator of hypertriglyceridemia, vascular disease, artherosclerotic disease and related conditions.

Cardiovascular disease (CVD) is a broad term that encompasses a variety of diseases and conditions. It refers to any disorder in any of the various parts of the cardiovascular system, which consists of the heart and all of the blood vessels found throughout the body. Diseases of the heart may include coronary artery disease, CHD, cardiomyopathy, valvular heart disease, pericardial disease, congenital heart disease (e.g., coarctation, atrial or ventricular septal defects), and heart failure. Diseases of the blood vessels may include arteriosclerosis, atherosclerosis, hypertension, stroke, vascular dementia, aneurysm, peripheral arterial disease, intermittent claudication, vasculitis, venous incompetence, venous thrombosis, varicose veins, and lymphedema. Some patients may have received treatment for their CVD, such as vascular or coronary revascularizations (angioplasty with or without stent placement, or vascular grafting). Some types of cardiovascular disease are congenital, but many are acquired later in life and are attributable to unhealthy habits, such as a sedentary lifestyle and smoking. Some types of CVD can also lead to further heart problems, such as angina, major adverse cardiovascular events (MACEs) and/or major coronary events (MCEs) such as myocardial infarction (MI) or coronary intervention, or even death (cardiac or cardiovascular), which underscores the importance of efforts to treat and prevent CVD.

Primary prevention efforts are focused on reducing known risk factors for CVD, or preventing their development, with the aim of delaying or preventing the onset of CVD, MACEs or MCEs. Secondary prevention efforts are focused on reducing recurrent CVD and decreasing mortality, MACEs or MCEs in patients with established CVD.

MACEs include cardiac death, other cardiovascular death, MCEs (which include myocardial infarction (MI) and coronary intervention such as coronary revascularization, angioplasty, percutaneous transluminal coronary angioplasty (PTCA), percutaneous coronary intervention (PCI) and coronary artery bypass graft (CABG)), hospitalization for unstable angina, stroke, transient ischemic attack (TIA) and hospitalization for peripheral artery disease (PAD).

The Third Report of the National Cholesterol Education Program Expert Panel on Detection, Evaluation, and Treatment of High Blood Cholesterol in Adults, NIH Publication No. 02-5215 (September 2002) (also known as the “NCEP ATP III”), hereby incorporated by reference, provides recommendations for cholesterol-lowering therapy in an effort to reduce risk of CHD. In the ATP III, CHD is defined as symptomatic ischemic heart disease, including MI, stable or unstable angina, demonstrated myocardial ischemia by noninvasive testing, and history of coronary artery procedures. The ATP III indicates that LDL-C is the primary target of lipid therapy, with other lipids to be controlled including triglycerides (TG), non-HDL-C and HDL-C.

A guiding principle of ATP III is that the intensity of LDL-C lowering therapy is adjusted to the individual's absolute risk for CHD. Risk assessment is broken down into short term (≦10-year) and long term (>10-year) risk of CHD, and the LDL-C goals are adjusted accordingly. In addition, ATP III identifies three categories of risk for CHD that modify LDL-C goals: established CHD and CHD risk equivalents, multiple (2+) risk factors, and 0-1 risk factor. Established CHD and CHD risk equivalents include CHD, other clinical atherosclerotic diseases, diabetes mellitus, and multiple risk factors and a 10-year risk for CHD>20 percent. The major independent risk factors identified in risk factor counting include cigarette smoking, hypertension, low HDL-C, family history of premature CHD and age.

The LDL-C goals for the three categories of risk factors are as follows:

| Risk Factors | LDL-C Goal |
|---|---|
| CHD and CHD Risk Equivalent | <100 mg/dl |
| Multiple (2+) Risk Factors | <130 mg/dl* |
| 0-1 Risk Factor | <160 mg/dl |

*LDL-C goal for multiple risk factor persons with 10-year risk >20 percent is <100 mg/dl.

The ATP III also outlines LDL-C goals for patients based on the percentage of 10-year risk for CHD:

| 10-Year Risk | LDL-C Goal |
|---|---|
| >20% | <100 mg/dl |
| 10-20% | <130 mg/dl |
| <10% and Multiple (2+) Risk Factors | <130 mg/dl |
| <10% and 0-1 Risk Factor | <160 mg/dl |

3-hydroxy-3-methyl glutaryl coenzyme A (HMG-CoA) reductase inhibitors (known as HMG-CoA inhibitors, or “statins”), have been used to treat hyperlipidemia and atherosclerosis, for example. Typically, statin monotherapy has been used to treat cholesterol levels, particularly when a patient is not at an acceptable LDL-C level. Statins inhibit the enzyme HMG-CoA reductase, which controls the rate of cholesterol production in the body. Statins lower cholesterol by slowing down the production of cholesterol and by increasing the liver's ability to remove the LDL-C already in the blood. Accordingly, the major effect of the statins is to lower LDL-C levels. Statins have been shown to decrease CHD risk by about one-third. However, statins only appear to have a modest effect on the TG-HDL axis.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII phospholipid complex. Further, omega-3 fatty acids seem to be well tolerated, without giving rise to any severe side effects.

One such form of omega-3 fatty acids is a concentrate of omega-3, long chain, polyunsaturated fatty acids from fish oil containing DHA and EPA and is sold under the trademark Lovaza® (formerly sold under the trademark Omacor®). Such a form of omega-3 fatty acids is described, for example, in U.S. Pat. Nos. 5,502,077, 5,656,667 and 5,698,594, each incorporated herein by reference.

In many patients with hypertriglyceridemia, hypercholesterolemia or mixed dyslipidemia, the use of diet and single-drug therapy does not always decrease LDL-C and TG adequately enough to reach targeted values. In these patients, a complementary combination therapy of a statin and omega-3 fatty acids may be desirable.

Many studies have examined the effects of fish oil and statin therapy, including: Nakamura et al., Int. J. Clin. Lab Res. 29:22-25 (1999); Davidson et al., Am. J. Cardiol. (1997) 80: 797-798; Hong et al., Chin. Med. Sci. J. 19:145-49 (2004); Contacos et al., Arterioscl. Thromb. 13:1755-62 (1993); Singer, Prost. Leukotr. Ess. Fatty Acids 72:379-80 (2005); Liu et al., Nutrition Research 23 (2003) 1027-1034; Grekas et al., Nephron (2001) 88: 329-333; Howe et al., Clin. Exp. Pharmacol. 29: A50-A51 (2002); Sandset et al., Arterioscler. Thromb. Vasc. Biol. 11:138-45 (1991); Tomei et al., Cardiologia, 38: 773-78 (1993); Nordoy et al., Essent. Fatty Acids Eicosanoids, Invited Pap. Int'l Congr. 3^{rd}, 252-56 (1992).

Studies have also investigated the effect of statins and Omacor® omega-3 fatty acids, including: Hansen et al., Arteriosclerosis and Thrombosis 14(2): 223-229 (February 1994); Nordoy et al., Nutr. Metab. Cardiovasc. Dis. (2001) 11:7-16; Salvi et al., Curr. Ther. Res. 53:717-21 (1993); Bhatnagar et al., Eur. Heart J Supplements (2001) 4 (Suppl. D): D53-D58; Chan et al., Diabetes, 51: 2377-2386 (August 2002); Chan et al., Eur. J of Clin. Invest. (2002) 32: 429-436; Nordoy et al., Essent. Fatty Acids Eicosanoids, Invited Pap. Int'l Congr. 4^{th}, 256-61 (1998); Nordoy et al., J. of Internal Medicine, 243:163-170 (1998); Durrington et al., Heart, 85:544-548 (2001).

Compositions comprising digestible oils, including in some instances omega-3 fatty acid oils, and another active ingredient have been disclosed. See, e.g., U.S. Pat. No. 6,096,338, U.S. Pat. No. 6,267,985, U.S. Pat. No. 6,667,064, U.S. Pat. No. 6,720,001, U.S. Patent Application Publication No. 2003/0082215, U.S. Patent Application Publication No. 2004/0052824, WO 99/29300, WO 2001/021154, U.S. Pat. No. 6,284,268, Saify et al., Pakistan J. of Pharm. Sci. (2003) 16(2): 1-8; Pennacchiotti et al., Lipids (2001) 26(2): 121-127; and Wysynski et al., Human and Experimental Toxicology (1993) 12: 337-340.

U.S. Patent Application Publication No. 2003/0170643 claims a method of treating a patient, by administering a therapeutic which lowers plasma concentrations of apoB and/or an apoB-containing lipoprotein and/or a component of an atherogenic lipoprotein by stimulating post-ER pre-secretory proteolysis (PERPP).

### SUMMARY OF THE INVENTION

The present invention is directed to a method of lipid therapy, comprising providing a subject group having a baseline triglyceride level of 200 to 499 mg/dl and being at or near its LDL-C treatment goal, and reducing the triglyceride level and the non-HDL-C level of the subject group as compared to treatment with an HMG CoA inhibitor alone, by administering to the subject group an effective amount of an HMG CoA inhibitor and a composition comprising omega-3 fatty acids.

In some embodiments, the HMG CoA inhibitor and the omega-3 fatty acids are administered as a single pharmaceutical composition as a combination product, for example, a unit dosage.

The present invention includes a combination product of an HMG CoA inhibitor and omega-3 fatty acids, in particular a combination product that provides a single administration of concentrated amounts of omega-3 fatty acids and an HMG CoA inhibitor, for example, in a unit dosage, and uses of such combination products in the methods of lipid therapy herein.

In variations of the present invention, the HMG CoA inhibitor is selected from the group consisting of pitavastatin, atorvastatin, rosuvastatin, fluvastatin, lovastatin, pravastatin and simvastatin. In particular embodiments, the HMG CoA inhibitor is simvastatin.

In one aspect of the invention, the methods of administration and/or the combination product is used in the treatment of one or more of the following: hypertriglyceridemia, hypercholesterolemia, mixed dyslipidemia, cardiovascular disease, vascular disease, atherosclerotic disease and related conditions, and/or for the prevention or reduction of cardiovascular and/or vascular events (including MACEs, MCEs).

Other features and advantages of the present invention will become apparent to those skilled in the art upon examination of the following or upon learning by practice of the invention.

### DESCRIPTION OF THE PREFERRED EMBODIMENTS

The present invention is directed to the utilization of HMG CoA inhibitors and omega-3 fatty acids for the treatment of one or more of the following: hypertriglyceridemia, hypercholesterolemia, mixed dyslipidemia, cardiovascular disease, vascular disease, atherosclerotic disease and related conditions, and/or for the prevention or reduction of cardiovascular and/or vascular events, and a combination product or unit dosage comprising one or more dyslipidemic agents and one or more omega-3 fatty acids.

In some embodiments, this invention provides a method of lipid therapy, comprising providing a subject group having a baseline triglyceride level of 200 to 499 mg/dl and being at or near its LDL-C treatment goal, and reducing the triglyceride level and the non-HDL-C level of the subject group as compared to treatment with an HMG CoA inhibitor alone, by administering to the subject group an effective amount of an HMG CoA inhibitor and a composition comprising omega-3 fatty acids.

In a preferred embodiment, the administration comprises omega-3 fatty acids, preferably in the form of the Lovaza® omega-3 fatty acids, and an HMG CoA inhibitor, wherein the omega-3 fatty acids are administered simultaneous to administration of the HMG CoA inhibitor, e.g., as a single fixed dosage pharmaceutical composition or as separate compositions administered at the same time.

In other preferred embodiments, the administration comprises omega-3 fatty acids and an HMG CoA inhibitor, wherein the omega-3 fatty acids are administered apart from the administration of the HMG CoA inhibitor, but in a concomitant treatment regime. For example, the HMG CoA inhibitor may be administered once daily while the omega-3 fatty acids are administered twice daily. One skilled in the art with the benefit of the present disclosure will understand that the precise dosage and schedule for the administration of the omega-3 fatty acids and the HMG CoA inhibitor will vary depending on numerous factors, such as, for example, the route of administration and the seriousness of the condition.

In some embodiments, the claimed method of administration is a first-line therapy, meaning that it is the first type of therapy given for the condition or disease. In other embodiments, the claimed method of administration is a second-line therapy, meaning that the treatment is given when initial treatment (first-line therapy, e.g., statin or omega-3 fatty acid treatment alone) does not work adequately with respect to treatment goals, or stops working adequately.

In some embodiments, the invention is suitable for primary prevention. In other embodiments, the invention is suitable for secondary prevention.

In preferred embodiments, the phrase “at or near its LDL-C treatment goal” means either at, below, or within 20% (preferably within 15%, most preferably within 10%) of the appropriate LDL-C goal set by a physician, preferably applying the standards set forth in the NCEP ATP III. The basic requirement for patients to be at or near their LDL-C goal is important because the widely accepted ATP III guidelines state that managing LDL-C should be the primary treatment objective in patients with dyslipidemia. As noted above, in the ATP III, the LDL-C goal can be calculated based on risk factors, the percentage of 10-year risk for CHD, or a combination of these elements. While the standards set forth in the ATP III are the currently preferred embodiment of the invention, this invention can also be applied using other standards of care and/or updated versions of the NCEP ATP.

In some embodiments, the subject group has a baseline LDL-C of or about 65 mg/dl, ≧ or about 70 mg/dl, ≧ or about 75 mg/dl, ≧ or about 80 mg/dl, ≧ or about 85 mg/dl, ≧ or about 90 mg/dl, ≧ or or about 95 mg/dl. In some particular embodiments, the subject group has such baseline LDL-C, to <160 (+/−20, 15 or 10%), <130 (+/−20, 15 or 10%) or <100 (+/−20, 15 or 10%) mg/dl. For example, the subject group has a baseline LDL-C of from ≧ or about 65 mg/dl to <160 (+/−20, 15 or 10%), <130 (+/−20, 15 or 10%) or <100 (+/−20, 15 or 10%) mg/dl.

In some embodiments, the subject group has a baseline LDL-C of ≧100 mg/dl, e.g. from ≧100 to <160 (+/−20, 15 or 10%) or <130 (+/−20, 15 or 10%) mg/dl.

In preferred embodiments, the selected subject group was receiving HMG CoA inhibitor therapy prior to the combination therapy of the HMG CoA inhibitor and the omega-3 fatty acids. Other active agents (other than omega-3 fatty acids) may also have been employed prior to the combination therapy of the HMG CoA inhibitor and the omega-3 fatty acids.

In other preferred embodiments, the method of the invention further comprises reducing at least one additional level of the subject group independently selected from the group consisting of the total cholesterol (TC) level, the apolipoprotein-B (Apo-B) level, and the very low-density lipoprotein cholesterol (VLDL-C) level, as compared to treatment with the HMG CoA inhibitor alone.

In other embodiments, the method of the invention further comprises increasing the high-density lipoprotein cholesterol (HDL-C) level, as compared to treatment with the HMG CoA inhibitor alone.

In other embodiments, the triglyceride level and the non-HDL-C level are reduced without increasing the LDL-C level, as compared to treatment with the HMG CoA inhibitor alone. In some embodiments, the triglyceride level and the non-HDL-C level are reduced without increasing LDL-C more than 1-6% (including ≦1, 2, 3, 4, 5, or 6%) as compared to treatment with the HMG CoA inhibitor alone. In some embodiments, the triglyceride level and the non-HDL-C level are reduced without increasing LDL-C more than 1% as compared to baseline.

The phrase “compared to treatment with an HMG CoA inhibitor alone” can refer to treatment of the same subject or subject group, or treatment of a comparable subject or subject group (i.e., subject(s) within the same class with respect to a particular blood protein, lipid, or marker, such as a cholesterol or triglyceride level) in a different treatment group. The terms “reduce” and “increase” in accordance with the claimed methods are intended to mean a statistically significant reduction or increase in accordance with its general and customary meaning, i.e., a probability of chance of 5% or less (p=0.05 or less), preferably p=0.025 or less. In some embodiments of the invention, the HMG CoA inhibitor alone statistically significantly reduces or increases certain levels (such as reducing triglyceride and/or LDL-C levels or increasing HDL-C levels), and the combination therapy of the HMG CoA inhibitor and the omega-3 fatty acids further statistically significantly reduces or increases the levels.

The methods and compositions of the invention may also be used to reduce any of the following blood protein, lipid, or marker levels in a treated subject or subject group, as compared to treatment with the HMG CoA inhibitor alone: RLP-C levels, Lp-PLA2 levels and/or Apo-C3 levels.

Preferably, non-HDL-C levels may be reduced at least about 5%, preferably at least about 7%, from baseline and/or at least about 5%, preferably at least about 7%, further than treatment with the HMG CoA inhibitor alone.

Preferably, the triglyceride levels may be reduced by at least about 20%, preferably at least about 25%, as compared to baseline and/or at least about 15%, preferably at least about 20%, further than treatment with the HMG CoA inhibitor alone.

Preferably, the VLDL-C levels may be reduced by at least about 20%, preferably at least about 25%, as compared to baseline and/or at least about 15%, preferably at least about 20%, further than treatment with the HMG CoA inhibitor alone.

Preferably, the TC levels may be reduced by at least about 3%, preferably at least about 5%, as compared to baseline and/or at least about 2%, preferably at least about 3%, further than treatment with the HMG CoA inhibitor alone.

Preferably, the RLP-C levels may be reduced by at least about 20%, preferably at least about 25%, as compared to baseline and/or at least about 15%, preferably at least about 20%, further than treatment with the HMG CoA inhibitor alone.

Preferably, the Lp-PLA2 levels may be reduced by at least about 7%, preferably at least about 10%, as compared to baseline and/or at least about 5%, preferably at least about 7%, further than treatment with the HMG CoA inhibitor alone.

Preferably, the Apo-B levels may be reduced by at least about 3%, preferably at least about 4%, as compared to baseline and/or at least about 1%, preferably at least about 2%, further than treatment with the HMG CoA inhibitor alone.

Preferably, the Apo-C3 levels may be reduced by at least about 5%, preferably at least about 7%, as compared to baseline and/or at least about 8%, preferably at least about 10%, further than treatment with the HMG CoA inhibitor alone.

Preferably, the HDL-C levels may be increased by at least about 2%, preferably at least about 3%, as compared to baseline and/or at least about 3%, preferably at least about 5%, further than treatment with the HMG CoA inhibitor alone.

Preferably, the present invention also decreases the ratio of TC to HDL-C, preferably by at least about 5%, more preferably at least about 10%, as compared to baseline and/or at least about 5%, preferably at least about 10%, further than treatment with the HMG CoA inhibitor alone.

Generally, the effect of the HMG CoA inhibitor is dose dependent, i.e., the higher the dose, the greater the therapeutic effect. However, the effect of each HMG CoA inhibitor is different, and therefore the level of therapeutic effect of one HMG CoA inhibitor cannot be necessarily be directly correlated to the level of therapeutic effects of other HMG CoA inhibitors. However, those of ordinary skill in the art would understand the correct dosage to be given to a particular subject, based on experience and the seriousness of the condition.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and a-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Lovaza® omega-3 fatty acids (K85EE, Pronova Biocare A.S., Lysaker, Norway) and preferably comprises the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

The combination product of an HMG CoA inhibitor and omega-3 fatty acids may be administered in a capsule, a tablet, a powder that can be dispersed in a beverage, or another solid oral dosage form, a liquid, a soft gel capsule, a coated soft gel capsule (see U.S. application Ser. No. 11/716,020, hereby incorporated by reference) or other convenient dosage form such as oral liquid in a capsule, as known in the art. In some embodiments, the capsule comprises a hard gelatin. The combination product may also be contained in a liquid suitable for injection or infusion.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include coilloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

Excipients include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The combination product of an HMG CoA inhibitor and omega-3 fatty acids is aided by the solubility of the HMG CoA inhibitor in the omega-3 fatty acid oil. Thus, the combination product does not require high amounts of solubilizers, such as surfactants, cosolvents, oils or combinations thereof. Preferably, the active ingredients are administered without the use of large amounts of solubilizers (other than the omega-3 fatty acid oil). In preferred embodiments, if present at all, solubilizers other than the omega-3 fatty acid oil are present in amounts of less than 50% w/w based on the total weight of the solvent system in the dosage form(s), preferably less than 40%, more preferably less than 30%, even more preferably less than 20%, still more preferably less than 10% and most preferably less than 5%. In some embodiments, the solvent system contains no solubilizers other than the omega-3 fatty acid oil. As used herein, “solvent system” includes the omega-3 fatty acid oil. In other preferred embodiments, the weight ratio of omega-3 fatty acid oil to other solubilizer is at least 0.5 to 1, more preferably at least 1 to 1, even more preferably at least 5 to 1, and most preferably at least 10 to 1.

In other preferred embodiments, if present at all, the amount of hydrophilic solvent used in the solvent system is less than 20% w/w based on the total weight of the solvent system in the dosage form(s), more preferably less than 10%, and most preferably less than 5%. In certain embodiments, the amount of hydrophilic solvent used in the solvent system is between 1 and 10% w/w.

In preferred embodiments, omega-3 fatty acid oil is present in amounts of at least 30% w/w based on the total weight of the solvent system in the dosage form(s), more preferably at least 40%, even more preferably at least 50%, and most preferably at least 60%. In certain embodiments, the amount can be at least 70%, at least 80% or at least 90%.

The dosage form is stable at room temperature (about 23° C. to 27° C.) for a period of at least one month, preferably at least six months, more preferably at least one year, and most preferably at least two years. By “stable”, applicants mean that the solubilized HMG CoA inhibitor does not come out of solution to any appreciable degree, for example, in amounts of less than 10%, preferably less than 5%.

The omega-3 fatty acids can be administered in a daily amount of from about 0.1 g to about 10 g, more preferably about 1 g to about 8 g, and most preferably from about 2 g to about 6 g. In one embodiment, the omega-3 fatty acids are administered in an amount up to 4 g/day.

The HMG CoA inhibitor may be administered in an amount more than, equal to or less than the conventional full-strength dose as a single-administered product. For example, the HMG CoA inhibitor may be administered in an amount of from 10-100%, preferably about 25-100%, most preferably about 50-80%, of the conventional full-strength dose as a single-administered product. In one embodiment of the present invention, the HMG CoA inhibitor can generally be present in an amount from about 0.5 mg to 80 mg, more preferably from about 1 mg to about 40 mg, and most preferably from about 5 mg to about 20 mg, per gram of omega-3 fatty acids. The daily dose may range from about 1 mg to about 320 mg, preferably about 2 mg to about 160 mg.

In some variations of the present invention, the combination of HMG CoA inhibitor and the omega-3 fatty acids is formulated into a single administration or unit dosage. In some embodiments, the HMG CoA inhibitor is selected from the group: atorvastatin, rosuvastatin, fluvastatin, lovastatin, pravastain, and simvastatin.

Pravastatin, which is known in the market as Pravachol® manufactured by Bristol-Myers Squibb, Princeton, N.J., is hydrophilic. Pravastatin is best absorbed without food, i.e., an empty stomach. The dosage of pravastatin, in the combined administration of omega-3 fatty acids is preferably from 2.5 to 80 mg, preferably 5 to 60, and more preferably from 10 to 40 mg per dosage of omega-3 fatty acids. In one variation, the combination product using pravastatin is taken at or around bedtime, e.g., 10 pm.

Lovastatin, which is marketed under the name Mevacor® by Merck, Whitehouse Station, N.J., is hydrophobic. Unlike pravastatin, lovastatin should be taken with meals and accordingly, in some embodiments, the combination product of omega-3 fatty acids and lovastatin should be taken with food. The dosage of lovastatin, in the combined administration of omega-3 fatty acids is preferably from 2.5 to 100 mg, preferably 5 to 80 mg, and more preferably from 10 to 40 mg per dosage of omega-3 fatty acids.

Simvastatin, which is marketed under the name Zocor® by Merck, Whitehouse Station, N.J., is hydrophobic. The dosage of simvastatin, in the combined administration of omega-3 fatty acids is preferably from 1 to 80 mg per day, preferably 2 to 60 mg, and more preferably from 5 to 40 mg per dosage of omega-3 fatty acids.

Atorvastatin, which is marketed under the name Lipitor® by Pfizer, New York, N.Y., is hydrophobic and is known as a synthetic statin. The dosage of atorvastatin, in the combined administration of omega-3 fatty acids is preferably from 2.5 to 100 mg, preferably 5 to 80 mg, and more preferably from 10 to 40 mg per dosage of omega-3 fatty acids.

Fluvastatin, which is marketed under the name Lescol® by Novartis, New York, N.Y., is hydrophilic and is known as a synthetic statin. The dosage of fluvastatin, in the combined administration of omega-3 fatty acids is from 5 to 160 mg, preferably 10 to 120 mg, and more preferably from 20 to 80 mg per dosage of omega-3 fatty acids.

Rosuvastatin is marketed under the name Crestor® by Astra Zeneca, Wilmington, Del. The dosage of rosuvastatin, in the combined administration of omega-3 fatty acids is from 1 to 80 mg, preferably 2 to 60 mg, and more preferably from 5 to 40 mg per dosage of omega-3 fatty acids.

Pitavastatin is currently marketed in Japan. The dosage of pitavastatin, in the combined administration of omega-3 fatty acids is from 0.25 to 20 mg, preferably 0.5 to 10 mg, and more preferably from 1 to 7.5 mg per dosage of omega-3 fatty acids.

The daily dosages of HMG CoA inhibitor and omega-3 fatty acids can be administered together in from 1 to 10 dosages, with the preferred number of dosages from 1 to 4 times a day, most preferred 1 to 2 times a day. The administration is preferably oral administration, although other forms of administration that provides a unit dosage of HMG CoA inhibitor and omega-3 fatty acids may be used.

In some embodiments, the formulations of the present invention allow for improved effectiveness of each active ingredient, with one or both administered as a conventional full-strength dose, as compared to the formulations in the prior art. In other embodiments, the formulations of the present invention may allow for reduced dosages of HMG CoA inhibitor and/or omega-3 fatty acids, as compared to the formulations in the prior art, while still maintaining or even improving upon the effectiveness of each active ingredient.

The present combination of a HMG CoA inhibitor and concentrated omega-3 fatty acids may allow for a greater effect than any expected combined or additive effect of the two drugs alone.

Thus, the combined treatment of the two active ingredients, separately or through the novel combination product of the present invention, may cause an unexpected increase in effect of the active ingredients that allows increased effectiveness with standard dosages or maintained effectiveness with reduced dosages of the two active ingredients. It is well accepted in practice that an improved bioavailability or effectiveness of a drug or other active ingredient allows for an appropriate reduction in the daily dosage amount. Any undesirable side effects may also be reduced as a result of the lower dosage amount and the reduction in excipients (e.g., surfactants).

### EXAMPLES

### Example 1

### Clinical Study: A Randomized, Double-Blind, Placebo-Controlled Study to Assess the Efficacy and Safety of Combined Omacor® and Simvastatin Therapy in Hypertriglyceridemic Subjects

A randomized, double-blind, placebo-controlled clinical study was conducted to assess the efficacy and safety of combined treatment with Omacor® omega-3 fatty acids and simvastatin (Zocor®) in hypertriglyceridemic subjects. Patients were initially treated with 40 mg/day simvastatin for at least 8 weeks, whereupon baseline measurements were taken. Patients were eligible for enrollment and randomization if their median baseline triglyceride levels were between 200 and 499 mg/dl and their LDL-C≦10% above the NCEP ATP III goal. Initial treatment was thereafter followed by an additional 8 week treatment with either 4 g/day Omacor® omega-3 fatty acids or placebo, while continuing statin therapy, in a double-blind fashion. 243 patients completed the study.

The following Table 1 shows the results obtained for changes in various lipid and inflammatory parameters and markers.

TABLE 1

| | Omacor | Placebo | | |
|---|---|---|---|---|
| | treatment: | treatment: | Difference | |
| | median % change | median % change | (% | |
| | from baseline | from baseline | median) | p-value |
| Non-HDL-C | −9.0% | −2.2% | −6.8% | <0.0001 |
| LDL-C | +0.7% | −2.8% | +3.5% | 0.0522 |
| Apo-B | −4.2% | −1.9% | −2.3% | 0.0232 |
| TG | −29.5% | −6.3% | −23.2% | <0.0001 |
| VLDL-C | −27.5% | −7.2% | −20.3% | <0.0001 |
| total C | −4.8% | −1.7% | −3.1% | 0.0013 |
| HDL-C | +3.4% | −1.2% | +4.6% | <0.0001 |
| TC/HDL | −9.6% | −0.7% | −8.9% | <0.0001 |
| RLP-C | −36.0% | −10.6% | −25.4% | <0.0001 |
| Lp-PLA2 | −12.8% | −4.7% | −8.1% | 0.0019 |
| Apo-C3 | −7.8% | +3.9% | −11.7% | 0.0002 |

The following Tables 2 and 3 show the LDL-C goal achievement experienced in the study by those on Omacor® treatment and placebo, respectively.

TABLE 2

| | | | End of Treatment | |
|---|---|---|---|---|
| Omacor | | | At or | |
| treatment | Baseline | | below goal | Above goal |
| | At or below | 113 (92.62%) | 110 (97.35%) | 3 (2.65%) |
| | goal | | | |
| | Above goal | 9 (7.38%) | 3 (33.33%) | 6 (66.67%) |
| | Total | 122 (100%) | 113 (92.62%) | 9 (7.38%) |

TABLE 3

| | | | End of Treatment | |
|---|---|---|---|---|
| Placebo | | | At or | |
| treatment | Baseline | | below goal | Above goal |
| | At or below | 120 (90.91%) | 117 (97.50%) | 3 (2.50%) |
| | goal | | | |
| | Above goal | 12 (9.09%) | 2 (16.67%) | 10 (83.33%) |
| | Total | 132 (100%) | 119 (90.15%) | 13 (9.85%) |

Table 4 shows a further analysis of certain lipid response in patients with baseline LDL-C>=100 mg/dL and in patients with baseline LDL-C<100 mg/dL (median % change from baseline values).

TABLE 4

| | Baseline LDLc <100 mg/dL | | | Baseline LDLc ≧100 mg/dL | | |
|---|---|---|---|---|---|---|
| | Omacor | placebo | | Omacor | placebo | |
| | (n = 87) | (n = 89) | Difference | (n = 35) | (n = 43) | Difference |
| Non-HDL-C | −8.6 | −2.5 | −6.1 | −10.2 | −1.5 | −8.7 |
| | | | p = 0.0002 | | | p = 0.0005 |
| TG | −29.1 | −7.0 | −22.1 | −29.8 | −1.8 | −28.0 |
| | | | p < 0.0001 | | | p < 0.0001 |
| Total C | −4.7 | −1.7 | −3.0 | −6.2 | −1.6 | −4.6 |
| | | | p = 0.0289 | | | p = 0.0066 |
| VLDL-C | −27.5 | −7.8 | −19.7 | −26.2 | −6.2 | −20.0 |
| | | | p < 0.0001 | | | p < 0.0001 |
| LDL-C | 2.4 | −1.8 | +4.2 | −5.4 | −3.9 | −1.5 |
| | | | p = 0.0108 | | | p = 0.6503 |
| HDL-C | 3.3 | −0.9 | +4.2 | 3.6 | −1.3 | +4.9 |
| | | | p < 0.0001 | | | p = 0.0218 |
| Apo-A1 | −1.9 | −0.7 | −1.2 | −2.7 | −1.7 | −1.0 |
| | | | p = 0.8117 | | | p = 0.5257 |
| Apo-B | −3.2 | −2.8 | −0.4 | −5.6 | −0.2 | −5.4 |
| | | | p = 0.4220 | | | p = 0.0016 |
| Lp-PLA2 | −12.6 | −2.8 | −9.8 | −13.4 | −8.9 | −4.5 |
| | | | p = 0.0108 | | | p = 0.0442 |

The following Table 5 shows the median change (CFB) and median percent change from baseline (% CFB) in various lipid and lipoprotein markers for subgroups according to tertile of baseline LDL-C.

TABLE 5

| | | Tertile 1 | | Tertile 2 | | Tertile 3 | |
|---|---|---|---|---|---|---|---|
| | | n = 84 | | n = 86 | | n = 84 | |
| | | Omacor | placebo | Omacor | placebo | Omacor | placebo |
| | | bl LDL-C | bl LDL-C | bl LDL-C | bl LDL-C | bl LDL-C | bl LDL-C |
| | | 31.7-80.3 mg/dL | 42.0-79.7 mg/dL | 82.0-98.7 mg/dL | 80.7-98.7 mg/dL | 99.0-145.3 mg/dL | 99.0-160.0 mg/dL |
| Parameter | Median | (n = 43) | (n = 41) | (n = 40) | (n = 46) | (n = 39) | (n = 45) |
| Non-HDL-C | CFB (mg/dL) | −6.0 | −0.2 | −16.6 | −4.3 | −15.3 | −2.8 |
| | % CFB | −5.4 | −0.2 | −12.6 | −3.6 | −10.8 | −1.6 |
| LDL-C | CFB (mg/dL) | 6.3 | 0.7 | −0.8 | −3.3 | −6.8 | −5.7 |
| | % CFB | 9.5^{a} | 1.1^{d} | −0.9^{b} | −3.8^{e} | −6.4^{c} | −4.5^{f} |
| VLDL-C | CFB (mg/dL) | −14.8 | −3.7 | −14.3 | −5.5 | −14.7 | −4.1 |
| | % CFB | −26.5 | −7.2 | −27.6 | −10.0 | −28.9 | −6.6 |
| Total Chol | CFB (mg/dL) | −3.3 | 0.5 | −14.4 | −5.3 | −12.3 | −4.2 |
| | % CFB | −2.2 | 0.3 | −7.8 | −2.6 | −6.2 | −2.0 |
| HDL-C | CFB (mg/dL) | 2.0 | −0.2 | 0.9 | −0.5 | 1.7 | −0.7 |
| | % CFB | 4.2 | −0.5 | 2.2 | −1.2 | 3.6 | −1.3 |
| TG | CFB (mg/dL) | −89.0 | −18.8 | −80.4 | −14.3 | −82.5 | −16.8 |
| | % CFB | −26.9 | −8.3 | −32.1 | −4.6 | −29.8 | −5.5 |

^{a}median baseline LDL was 69 (range 31.7 to 80.3); % change LDL range −18.6 to 113.1
^{b}median baseline LDL was 91.3 (range 82 to 98.7); % change LDL range −21.2 to 25.8
^{c}median baseline LDL was 109 (range 99 to 145.3); % change LDL range −33.3 to 31.6
^{d}median baseline LDL was 72.7 (range 42 to 79.7); % change LDL range −29.6 to 33.3
^{e}median baseline LDL was 86.8 (range 80.7 to 98.7); % change LDL range −32 to 19.8
^{f}median baseline LDL was 112.3 (range 99 to 160); % change LDL range −29.8 to 22.4

An increase in the median % CFB in LDL-C was observed among only those with baseline LDL-C in the lowest tertile, which had a median baseline LDL-C of 69.

### Example 2

### Clinical Study: A Randomized, Double-Blind, Placebo-Controlled, Forced-Titration Study to Assess the Efficacy and Safety of OMACOR Co-Administered With Open-Label Atorvastatin Therapy, in Hypertriglyceridemic Subjects

A study was undertaken to examine the effects of OMACOR in combination with a statin in a population of subjects with mixed dyslipidemia. The combination of OMACOR+atorvastatin was given as initial (first-line) therapy. The effect of the combination of OMACOR and atorvastatin on non-HDL-C and other lipid and lipoprotein endpoints was examined across 3 sequential dosing periods with atorvastatin 10 mg, 20 mg, and 40 mg.

The study was a 20-week, multi-center, randomized, double-blind, placebo-controlled, parallel-group study with a 4-week, lead-in period and a 16-week, double-blind treatment period. During the double-blind treatment period, subjects were treated for 8 weeks with open-label atorvastatin (10 mg) and either OMACOR or placebo. After the initial 8-week treatment period, the dose of atorvastatin was increased to 20 mg for 4 weeks, followed by a second dose-escalation of atorvastatin to 40 mg for an additional 4 weeks.

Subjects meeting all study requirements after the screening visit (Visit 1, Week -4) entered a 4-week, diet-only, lead-in period, during which no lipid-lowering agents were used. Subjects received counseling regarding the National Cholesterol Education Program Therapeutic Lifestyle Changes diet. At the end of the lead-in period, subjects with a non-HDL-C level>160 mg/dL and TG levels ≧250 mg/dL and ≦599 mg/dL, and who met other entry criteria, were assigned randomly with stratification to receive either double-blind OMACOR 4 g+open-label atorvastatin 10 mg, or matching placebo +open-label atorvastatin 10 mg for 8 weeks. A stratified randomization scheme was used in order to ensure balance between the 2 treatment groups according to baseline TG concentration in each of the following strata: ≧250 mg/dL to ≦399 mg/dL or ≧400 mg/dL to ≦599 mg/dL. The non-HDL-C and TG levels were based on the average of the Week-2 (Visit 2) and Week -1 (Visit 3) values. In cases in which a subject's average non-HDL-C and/or TG level from Weeks -2 and -1 fell outside the required range for entry into the double-blind treatment period, 1 additional lipid panel could be collected. If a third sample was collected (designated as V3R), entry into the double-blind treatment period was based upon the average of values from Weeks −2, −1, and V3R for non-HDL-C and TG.

After the initial 8-week treatment period, the dose of atorvastatin was increased to 20 mg for an additional 4 weeks for all subjects. At Week 12 (Visit 9), the dose of atorvastatin was increased to 40 mg for an additional 4 weeks. Subjects who were unable to tolerate the increased dose of atorvastatin were discontinued from the study.

Treatments were:
OMACOR 1-gram capsule administered orally as 4-gram dose
Placebo capsule matching OMACOR 1-gram capsule administered orally as 4-gram dose,
Commercially available Lipitor® 10 mg tablet administered orally,
Commercially available Lipitor® 20 mg tablet administered orally,
Commercially available Lipitor® 40 mg tablet administered orally.

Table 6 shows percent changes in certain lipid parameters (mg/dL), apolipoproteins (mg/dL), lipid Ratios, Lp-PLA2 (nmol/L), and RLP-C (mg/dL) from baseline to endpoint of each Atorvastatin treatment period—Modified Intent-to-Treat Population (which included all randomized subjects who took at least 1 dose of study medication and provided at least 1 post-randomization efficacy data point).

TABLE 6

| | Omacor + Atorvastatin | | | Placebo + Atorvastatin | | | Treatment | |
|---|---|---|---|---|---|---|---|---|
| | (N = 122) | | | (N = 121) | | | Difference | |
| | | | Median | | | Median | Median | |
| | | Baseline [2] | Percent | | Baseline [2] | Percent | Percent | P-value |
| Parameter | n [1] | Median | Change | n [1] | Median | Change | Change | [3] |

10 mg Atorvastatin Treatment Period:

| | | | | | | | | |
|---|---|---|---|---|---|---|---|---|
| Non-HDL-C | 122 | 213.5 | −40.2 | 121 | 215.5 | −33.7 | −6.5 | 0.0002 |
| TC | 122 | 249.8 | −31.5 | 121 | 252.5 | −27.4 | −4.1 | 0.0015 |
| HDL-C | 122 | 36.0 | 12.4 | 121 | 37.5 | 10.0 | 2.4 | 0.0066 |
| LDL-C | 122 | 140.0 | −29.3 | 120 | 146.5 | −31.5 | 2.2 | 0.2416 |
| TG | 122 | 348.0 | −45.4 | 121 | 315.5 | −26.9 | −18.6 | <0.0001 |
| VLDL-C | 122 | 72.0 | −54.3 | 120 | 69.0 | −37.0 | −17.3 | <0.0001 |
| Apo A-I | 122 | 143.0 | −0.3 | 121 | 144.0 | 1.5 | −1.8 | 0.1710 |
| Apo B | 122 | 148.0 | −32.1 | 121 | 152.0 | −30.7 | −1.4 | 0.1314 |
| Apo C-III | 119 | 18.0 | −29.4 | 119 | 17.0 | −16.0 | −13.4 | <0.0001 |
| TC/HDL-C | 122 | 7.2 | −38.3 | 121 | 7.1 | −34.5 | −3.8 | <0.0001 |
| TG/HDL-C | 122 | 9.7 | −51.9 | 121 | 8.8 | −34.0 | −17.8 | <0.0001 |
| Lp-PLA2 | 119 | 176.0 | −20.7 | 118 | 172.5 | −9.7 | −11.0 | <0.0001 |
| RLP-C | 118 | 21.0 | −50.0 | 119 | 21.0 | −38.9 | −11.1 | <0.0001 |

20 mg Atorvastatin Treatment Period:

| | | | | | | | | |
|---|---|---|---|---|---|---|---|---|
| Non-HDL-C | 117 | 212.5 | −46.9 | 118 | 214.8 | −39.0 | −7.9 | <0.0001 |
| TC | 117 | 247.5 | −38.1 | 118 | 252.5 | −32.4 | −5.8 | <0.0001 |
| HDL-C | 117 | 36.0 | 14.3 | 118 | 37.8 | 8.5 | 5.8 | 0.0004 |
| LDL-C | 115 | 139.0 | −38.3 | 116 | 148.0 | −38.3 | 0.0 | 0.8560 |
| TG | 117 | 348.0 | −51.2 | 118 | 315.0 | −30.7 | −20.5 | <0.0001 |
| VLDL-C | 115 | 72.0 | −61.3 | 116 | 69.0 | −43.3 | −18.1 | <0.0001 |
| Apo A-I | 115 | 143.0 | 1.1 | 116 | 144.0 | 2.2 | −1.1 | 0.1433 |
| Apo B | 115 | 148.0 | −36.8 | 116 | 152.0 | −33.6 | −3.2 | 0.0052 |
| Apo C-III | 115 | 18.0 | −31.8 | 116 | 16.5 | −20.0 | −11.8 | <0.0001 |
| TC/HDL-C | 117 | 7.2 | −45.1 | 118 | 6.9 | −37.5 | −7.7 | <0.0001 |
| TG/HDL-C | 117 | 9.8 | −57.4 | 118 | 8.6 | −38.4 | −19.0 | <0.0001 |
| Lp-PLA2 | 115 | 176.0 | −23.2 | 115 | 171.0 | −15.3 | −7.9 | <0.0001 |
| RLP-C | 114 | 21.0 | −61.8 | 116 | 21.5 | −50.0 | −11.8 | <0.0001 |

40 mg Atorvastatin Treatment Period:

| | | | | | | | | |
|---|---|---|---|---|---|---|---|---|
| Non-HDL-C | 113 | 210.0 | −50.4 | 112 | 214.8 | −46.3 | −4.1 | 0.0007 |
| TC | 113 | 246.5 | −41.1 | 112 | 252.5 | −38.4 | −2.7 | 0.0257 |
| HDL-C | 113 | 36.0 | 12.7 | 112 | 37.5 | 6.3 | 6.3 | <0.0001 |
| LDL-C | 112 | 140.0 | −43.4 | 111 | 146.0 | −43.7 | 0.3 | 0.6387 |
| TG | 113 | 348.0 | −53.9 | 112 | 315.0 | −34.8 | −19.1 | <0.0001 |
| VLDL-C | 112 | 72.0 | −65.2 | 111 | 68.0 | −47.8 | −17.4 | <0.0001 |
| Apo A-I | 113 | 143.0 | −1.4 | 112 | 143.5 | −0.3 | −1.1 | 0.5279 |
| Apo B | 113 | 148.0 | −41.3 | 112 | 151.0 | −38.7 | −2.6 | 0.2071 |
| Apo C-III | 113 | 18.0 | −35.3 | 112 | 16.0 | −22.2 | −13.1 | <0.0001 |
| TC/HDL-C | 113 | 7.2 | −48.1 | 112 | 7.0 | −41.6 | −6.5 | <0.0001 |
| TG/HDL-C | 113 | 9.8 | −60.9 | 112 | 8.6 | −39.4 | −21.5 | <0.0001 |
| Lp-PLA2 | 113 | 176.0 | −24.7 | 111 | 171.0 | −19.7 | −5.1 | 0.0072 |
| RLP-C | 112 | 21.0 | −61.7 | 112 | 21.5 | −52.1 | −9.6 | <0.0001 |

[1.] n is the number of subjects with values at both baseline and endpoint.
[2.] Baseline for non-HDL-C, TC, HDL-C, and TG was the average of Visit 3 (Week −1), V3R (if applicable), and Visit 4 (Week 0) values. If a subject's average non-HDL-C level and/or TG from Week-2 and Week-1 fell outside the required range, 1 additional lipid panel could be collected (designated as V3R). Baseline for LDL-C, VLDL-C, apo A-I, apo B, apo C-III, Lp-PLA2, and RLP-C was the Visit 4 (Week 0) value.
[3.] The p-value for comparison was from an analysis of variance model with rank as the response variable and treatment group and baseline TG stratum as independent variables.
Apo A-I = apolipoprotein A-I;
Apo B = apolipoprotein B;
Apo C-III = apolipoprotein C-III;
HDL-C = high-density lipoprotein cholesterol;
LDL-C = low-density lipoprotein cholesterol;
Lp-PLA2 = lipoprotein-associated phospholipase A2;
non-HDL-C = non-high-density lipoprotein cholesterol;
RLP-C = remnant-like particle cholesterol;
TC = total cholesterol;
TG = triglycerides;
VLDL-C = very-low-density lipoprotein cholesterol.

The following formulations may be prepared in accordance with the invention:

Formulation 1:

| Ingredient | Mg/capsule |
|---|---|
| K85EE | 1000 |
| Dehydrated ethanol | 39.5 |
| Capmul ® MCM | 20 |
| Simvastatin | 20 |

Formulation 2:

| Ingredient | Mg/capsule |
|---|---|
| EPAX7010EE | 1000 |
| Propylene glycol | 30 |
| Olive oil | 50 |
| Atorvastatin | 10 |

All references cited herein are hereby incorporated by reference in their entirety.

We claim:

1. A composition for lipid therapy in a subject comprising a fixed dosage form comprising an HMG CoA inhibitor and natural or synthetic omega-3 fatty acids or pharmaceutically acceptable esters, derivatives, conjugates, precursors or salts thereof, or mixtures thereof.

2. The composition of claim 1, wherein the composition comprises a solvent system including solubilizers in amounts of less than 25% w/w based on the total weight of the solvent system.

3. The composition of claim 1, wherein the composition comprises a solvent system including solubilizers in amounts of less than 10% w/w based on the total weight of the solvent system.

4. The composition of claim 1, wherein the composition comprises a solvent system including no solubilizers other than the omega-3 fatty acids.

5. The composition of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

6. The composition of claim 1, wherein the omega-3 fatty acids are present in a concentration of at least 40% by weight as compared to the total fatty acid content of the composition.

7. The composition of claim 1, wherein the omega-3 fatty acids are present in a concentration of at least 80% by weight as compared to the total fatty acid content of the composition.

8. The composition of claim 1, wherein the omega-3 fatty acids comprise at least 50% by weight of EPA and DHA as compared to the total fatty acid content of the composition.

9. The composition of claim 1, wherein the omega-3 fatty acids comprise at least 80% by weight of EPA and DHA as compared to the total fatty acid content of the composition.

10. The composition of claim 1, wherein the omega-3 fatty acids comprise about 5% to about 95% by weight of EPA as compared to the total fatty acid content of the composition.

11. The composition of claim 1, wherein the omega-3 fatty acids comprise about 40% to about 55% by weight of EPA as compared to the total fatty acid content of the composition.

12. The composition of claim 1, wherein the omega-3 fatty acids comprise about 5% to about 95% by weight of DHA as compared to the total fatty acid content of the composition.

13. The composition of claim 1, wherein the omega-3 fatty acids comprise about 30% to about 60% by weight of DHA as compared to the total fatty acid content of the composition.

14. The composition of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

15. The composition of claim 1, wherein the omega-3 fatty acids comprise EPA and DHA in a ratio of EPA:DHA from 99:1 to 1:99.

16. The composition of claim 1, wherein the omega-3 fatty acids comprise EPA and DHA in a ratio of EPA:DHA from 2:1 to 1:2.

17. The composition of claim 1, wherein the omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof, the omega-3 fatty acids are present in a concentration of at least 80% by weight of the composition, the omega-3 fatty acids comprise at least 80% by weight of EPA and DHA, about 40% to about 55% by weight of EPA, and about 30% to about 60% by weight of DHA as compared to the total fatty acid content of the composition, and the omega-3 fatty acids comprise EPA and DHA in a ratio of EPA:DHA from 2:1 to 1:2.

18. The composition of claim 17, wherein the omega-3 fatty acids are present in a concentration of at least 90% by weight of the composition.

19. The composition of claim 1, wherein the HMG CoA inhibitor comprises simvastatin.
